(19) United States
(12) Patent Application Publication
(10) Pub. No.: US 2023/0095862 A1
(43) Pub. Date: Mar. 30, 2023

(54) Supportive Compression Garment

(71) Applicant: Jessie M. HERZFELD ZIMMERMAN, Boise, ID (US)

(72) Inventor: Jessie M. HERZFELD ZIMMERMAN, Boise, ID (US)

(21) Appl. No.: 17/936,341

(22) Filed: Sep. 28, 2022

Related U.S. Application Data
(60) Provisional application No. 63/249,248, filed on Sep. 28, 2021.

(51) Int. Cl. A61F 5/02; A41D 13/12

(52) U.S. Cl. CPC A61F 5/028; A41D 13/1254; A41D 2500/10

(57) ABSTRACT

An improved support system for use with a lower body garment being a pants, short or skirt and preferably as a pair of pants for medical scrubs. The garment has a waistband that comprises a series of layers providing support and/or compression to the wearer. The waistband layer can extend to a typical pant material, or alternatively the pant material can be exterior to the waistband so as to cover the waistband. The supporting material provides additional support to the wearer, for example when standing for a long time such as in a shift of work in a hospital setting.

### PRIORITY/CROSS-REFERENCE TO RELATED APPLICATIONS

This application claims the benefit of U.S. Provisional Application No. 63/249,248, filed Sep. 28, 2021 the disclosure of which is incorporated by reference.

### TECHNICAL FIELD

The disclosure generally relates to the field of garments. Particular embodiments relate to supportive compression garments for the lower body.

### BACKGROUND

An issue that is common for people who spend an extended amount of time on their feet is back pain and/or strain, specifically in the lower back and/or abdominal region. This issue can be particularly prevalent in the field of medicine. To address such issue, some people may turn to wearing compression/support garments and/or support braces to support their backs and core.

Some types of lower body garments may be designed only to provide support to the lower back. In providing support to the lower back, several designs of lower body garments have been previously used. Some lower body garments may be designed from a compression material such that the compression fabric of the waistband area compresses and supports the lower back area.

Alternatively, lower body garments may be designed with an internal supportive belt structure to provide support to the lower back area of a wearer. The supportive belt may be integrated with the lower body garment. The belt may have opposing adjustable straps in the front of the garment such that a wearer can tighten the belt to provide the desired support.

Other lower body garment designs to provide support to the lower back area may also include a more rigid belt structure. This type of rigid belt may or may not be incorporated as part of the lower body garment. The belt structure may have added rigidity using plastic inserts or other more rigid materials in the rear portion of the belt to support the back.

Compression garments may also be used to address various other issues. For instance, compression garments may be used to provide a wearer with compression of the abdominal area to control excess abdominal weight. Compression fabric may be designed for other uses as well, such as for use in active wear apparel and/or to provide compressive support to a targeted area. Active wear compression garments may be designed to increase blood flow during and after an active activity. Active wear compression garments can also be designed such that the compression lessens loose hanging clothing and/or parts thereof.

Compression garments are typically made from a type of compression material. This material can include nylon, spandex, silicon, and/or cotton. The compression material in compression garments can also be made from a combination of the above listed materials to provide a desired texture and/or have specific characteristics.

Abdominal compression garments are typically designed to target abdominal weight and provide the desired compression to reduce the appearance of the abdominal weight. The compression that may be commonly used in garments to control abdominal weight can come in the form of a compressive elastic waistband. The elastic of the compressive waistband allows the waistband to comfortably conform to a wearer's body while still providing the desired compression to the abdominal area.

Some problems that may arise with abdominal compression garments is that they may give a wearer an unnatural look in the targeted area. The compression may squeeze or pinch the abdominal area such that it results unnatural bulging in certain areas while leaving other areas with the desired appearance. It may also lead to gastric issues if not considerate of anatomy, especially if the pressure is too tight. Additional problems include the support or bracing being too bulky and not functional.

Other examples of abdominal compression garments exist as well. For instance, abdominal compression garments may come in the form of an internal adjustable waistband that is covered by an external waistband. The internal waistband may be tightened by a wearer to provide the desired compression. The internal waistband can be covered by an external waistband that provides coverage of the internal waistband and compressed abdominal area. In these forms of compression garments, the internal compression waistband may be designed such that two opposing ends attach to one another in the front of the lower body garment. The attachment may be achieved via hook and loop fasteners. Other suitable means for attachment may also be used. The external waistband can come in the form of a traditional waistband where attachment of opposing sides is achieved by a plurality of buttons and/or a button and zipper combination.

### SUMMARY

The purpose of the Summary is to enable the public, and especially the scientists, engineers, and practitioners in the art who are not familiar with patent or legal terms or phraseology, to determine quickly from a cursory inspection, the nature and essence of the technical disclosure of the application. The Summary is neither intended to define the inventive concept(s) of the application, which is measured by the claims, nor is it intended to be limiting as to the scope of the inventive concept(s) in any way.

An object of this invention is to provide working professionals with a garment that is anatomically supportive of the front lower abdominal and lower back parts of the body in a functional, comfortable, and practical manner. Integrating this functionality into workwear eliminates the need to use a separate garment or brace to provide the necessary anatomical support. What is disclosed herein is a compression and support garment for the lower body. In particular, what is disclosed is a compression and support garment that can provide compression of the abdominal area and support to the lower back region. In a preferred embodiment the compression support garment is designed primarily as scrub bottoms for the working professional but may also apply to other professional uniforms. This invention is comprised of a supportive, multi-layer, mid-to-high waistband for scrubs and/or other professional uniforms. The waistband will be configured to provide support in the lower back and lower abdominal area. Compression will be utilized to provide for abdominal/core control and support.

What is disclosed is a multi layered waistband having a front side and a back side. The multi layered waistband has an inner layer and an outer layer. The multi layered waistband extends to standard leg covering (pants, shorts) or skirt or dress.

The inner layer of the multi layered waistband is comprised of a compression material. The inner layer has a compression and support structure, said compression and support structure. The compression and support structure is configured to provide compression and support to targeted areas of a wearer. The outer layer of the multi layered waistband is comprised of the same material as the remainder of said lower body garment. The outer layer and said lower body garment are made from an antimicrobial, moisture wicking material or acceptable material of similar nature.

The compression and support structure of the inner layer can be designed as an X pattern with the X pattern is configured to provide compression and support to the targeted areas.

The compression and support structure can include a lower compression and support structure configured to circumvolve said inner layer. The lower compression and support structure is configured to provide additional support to the targeted areas. The garment can include one or more vertical support structures. The compression and support structure of said inner layer can be designed with angled compressive support structures. The angled compressive support structures are configured to provide compression and support to the targeted areas.

The compression and support structure can be a lower compression and support structure that circumvolves the inner layer. The lower compression and support structure is configured to provide additional support to the targeted areas. Preferably a drawstring is used to provide additional support and/or to tighten one or more of the layers on the user's body.

Preferably the multi layered waistband has four layers being a waistband outside layer, a drawstring layer, a Waistband (Reinforced) layer, and a waistband lining layer. Preferably the lower body garment outer layer is a ribbed knit compression material, the drawstring layer being a neoprene and a compression material, the waistband layer being a neoprene, and the waistband lining layer comprises a 4-way stretch compression material.

Still other features and advantages of the presently disclosed and claimed inventive concept(s) will become readily apparent to those skilled in this art from the following detailed description describing preferred embodiments of the inventive concept(s), simply by way of illustration of the best mode contemplated by carrying out the inventive concept(s). As will be realized, the inventive concept(s) is capable of modification in various obvious respects all without departing from the inventive concept(s). Accordingly, the drawings and description of the preferred embodiments are to be regarded as illustrative in nature, and not as restrictive in nature

### BRIEF DESCRIPTION OF THE DRAWINGS

FIG. 1 is a front perspective view of an embodiment of the compression support garment.

FIG. 2 is rear perspective view of an embodiment of the compression support garment.

FIG. 3 is a perspective view of an embodiment of the inner layer of the multi layered waistband.

FIG. 4 is a perspective view of an embodiment of the inner layer of the multi layered waistband.

FIG. 5 is a perspective view of an embodiment of the inner layer of the multi layered waistband.

FIG. 6 is a perspective view of an embodiment of the inner layer of the multi layered waistband.

FIG. 7a is a view of the front of the outer layer and waistband layer.

FIG. 7b is a view of the back of the outer layer and waistband layer.

FIG. 8a is a view of the front section of a neoprene layer.

FIG. 8b is a view of a back section of the neoprene layer of 8a.

FIG. 9a is a view of a front section of an innermost layer.

FIG. 9b is a view of a back section of the innermost layer of FIG. 9a.

### DETAILED DESCRIPTION OF THE FIGURES

While the presently disclosed inventive concept(s) is susceptible of various modifications and alternative constructions, certain illustrated embodiments thereof have been shown in the drawings and will be described below in detail. It should be understood, however, that there is no intention to limit the inventive concept(s) to the specific form disclosed, but, on the contrary, the presently disclosed and claimed inventive concept(s) is to cover all modifications, alternative constructions, and equivalents falling within the spirit and scope of the inventive concept(s) as defined herein.

In the following description and in the figures, like elements are identified with like reference numerals. The use of “e.g.,” “etc,” and “or” indicates non-exclusive alternatives without limitation unless otherwise noted. The use of “including” means “including, but not limited to,” unless otherwise noted.

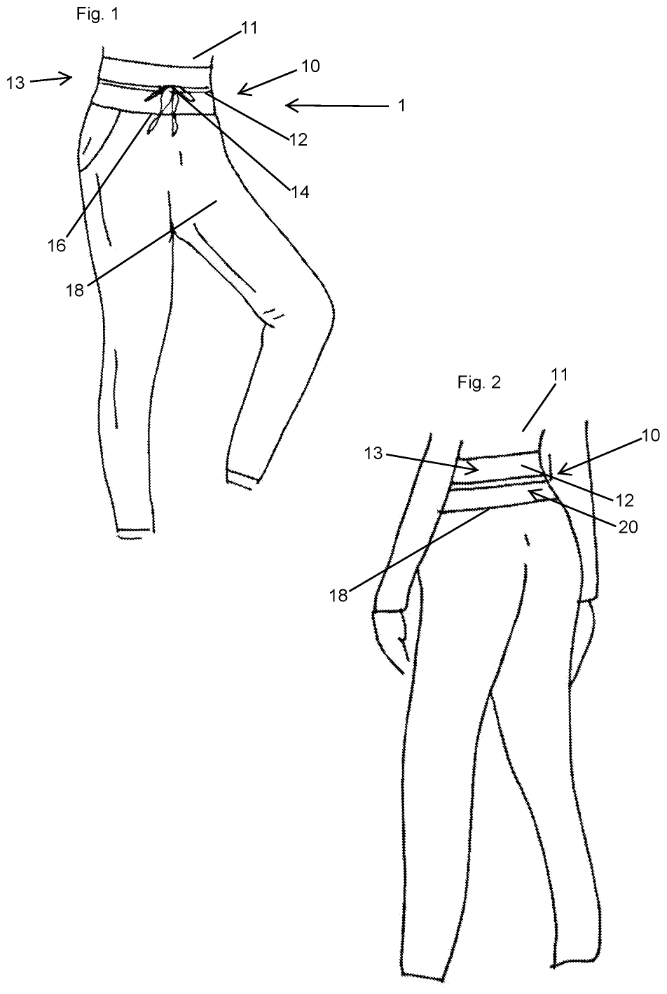

FIG. 1 is a front perspective view of an embodiment of the supportive compression garment for the lower body in use as a medical scrubs. The supportive compression garment 1 has a multi layer waistband 10. The multi layer waistband 10 is preferably designed to be a mid-to-high waistband. The mid-to-high waistband allows the multi layer waistband 10 to provide compression and support to targeted areas of a wearer. The multi layer waistband 10 has an outer layer 12. The outer layer 12 is comprised of the same or similar material as that of the lower body garment 18. In a preferred embodiment the outer layer 12 and the lower body garment 18 may be made from an antimicrobial and/or moisture wicking material. The multi layer waistband 10 can optionally include a drawstring 14.

The multi layer waistband 10 extends to the lower body garment 18 being looser fitting pants, specifically those found in medical scrubs. The multi layer waistband 10 may be attached to the lower body garment via stitching 16. Alternatively, the outer layer 12 of the multi layer waistband 10 may be designed such that it is formed of a single piece of material with the lower body garment 18. The multi layer waistband 10 is configured to provide support to the abdomen firmly at or above the umbilicus and extend downward to the greater trochanter or iliac crest. The multi layer waistband 10 spans horizontally around a wearer.

FIG. 2 is a rear perspective view of an embodiment of the supportive compression garment 1. The rear of the multi layer waistband 10 is attached to the lower body garment 18 in the same fashion as the front of the multi layer waistband. The rear of the multi layer waistband 12 has a support structure 20. The support structure 20 is configured to provide support to the lower back area of a wearer.

FIGS. 3 through 6 depict various embodiments of the inner layer 22 of the multi layer waistband 10. The inner layer 22 of the multi layer waistband 10 is comprised of a supportive compression material. The material of the inner layer 22 differs from the material of the outer layer 12. The supportive compression material can be a solid fabric material. Alternatively, the supportive compression material can be a mesh material designed to provide support and compression to a wearer. The inner layer 22 may be made from, for example, a nylon fabric, a spandex fabric, a silicon fabric, a neoprene, a polymer based fabric and/or a cotton fabric. Alternatively, the inner layer 22 may be made from a combination of the above listed fabrics. The inner layer 22 is attached to the outer layer 12 via stitching. Alternative methods of attaching the inner layer 22 to the outer layer 12 may also be used.

The inner layer 22 is configured to provide support and compression to a wearer in both the lower back area and the abdominal area. The back portion 23 of the inner layer 22 is configured to provide support to the lower back of a wearer. The back portion 23 of the inner layer 22 may be designed from various compression structures to provide the desired support and compression to the lower back. The inner layer 22 is configured to provide support between the greater trochanter and iliac crest bilaterally and spans in a horizontal direction to unite sacroiliac joint surfaces. Accessory support is oriented obliquely, inferior laterally and superiorly medialy. Support and compression begins at the gluteus and spans superiorly and medially to contralateral latissimus below the rib cage of a wearer. The inner layer 22 at the back portion 23 provides tension pulling towards a mid-point at the L5/S1 anatomical location.

The abdominal side of inner layer 22 is configured to provide support by pulling toward the midline of the trunk of the body of a wearer. The abdominal side of the inner layer spans in a horizontal direction from one anterior superior iliac spine to the other. The lateral tension of the abdominal side of the inner layer will decrease as the multi layer waistband 10 spans upward to the base of the ribs. This avoids excessive localized pressure on the viscera.

FIG. 3 depicts a prospective view of one embodiment of the back portion 23 of the inner layer 22. The embodiment of FIG. 3 depicts an opposing angled compression support structure 24. The angled compression support structure 24 is configured to provide support to the lower back area of a wearer. The back portion 23 may include a vertical support structure 28 positioned substantially in the middle of the back portion 23. The support structure 28 is configured to provide support along a wearer's spinal area. Alternatively, the inner layer 22 may be designed to include a plurality of vertical support structures 28 to provide additional support to the lower back area. The back portion 23 also includes a lower compression support structure 26. The lower compression support structure 26 is configured to provide additional support to a wearer's lower back area. The lower compression support structure 26 may also extend all the way around to the front of the inner lay 22. The lower compression support structure 26 in the front of the inner layer 22 is configured to provide compression in the abdominal area of a wearer.

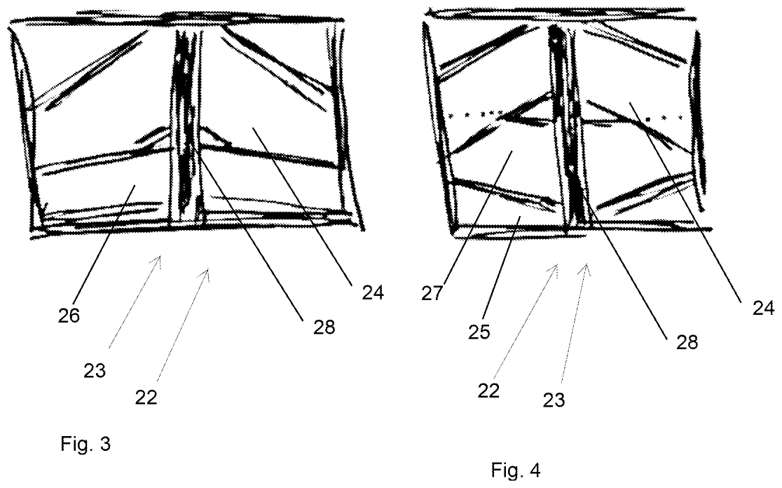

FIG. 4 depicts an alternative embodiment of the back portion 23 of the inner layer 22. The embodiment of FIG. 4 includes the angled compression support structure 24. There is an added intermediate compression support structure 27. The intermediate compression support structure 27 is configured to provide additional support and compression to the lower back area of a wearer. There is also an angled lower compression support structure 25. The angled lower compression support structure 25 is configured to provide additional support and compression to the lower back area of a wearer. The angled lower compression support 25 may also extend all the way around to the front of the inner layer 22. The angled lower compression support structure 25 is configured to provide compression in the abdominal area of a wearer.

FIGS. 5 and 6 depict alternative embodiments of the compression support structure of the inner layer 22. FIG. 5 depicts an integrated X patterned compression support structure 29. The integrated X patterned compression support structure 29 is configured to provide compression and support to the lower back area of a wearer. The abdominal side of the inner layer 22 has a corresponding integrated X patterned compression support structure. The integrated X patterned compression support structure of the abdominal side of the inner layer is also configured to provide compression and support of the abdominal area of a wearer. FIG. 6 depicts a compression support structure having a first member 30 and a second member 32. The first and second members 30, 32 are designed to create a cross weaved X 31. The cross weaved X 31 compression support structure is configured to provide compression and support to the lower back area of a wearer. The embodiments of FIGS. 5 and 6 include the vertical compression support structure 28.

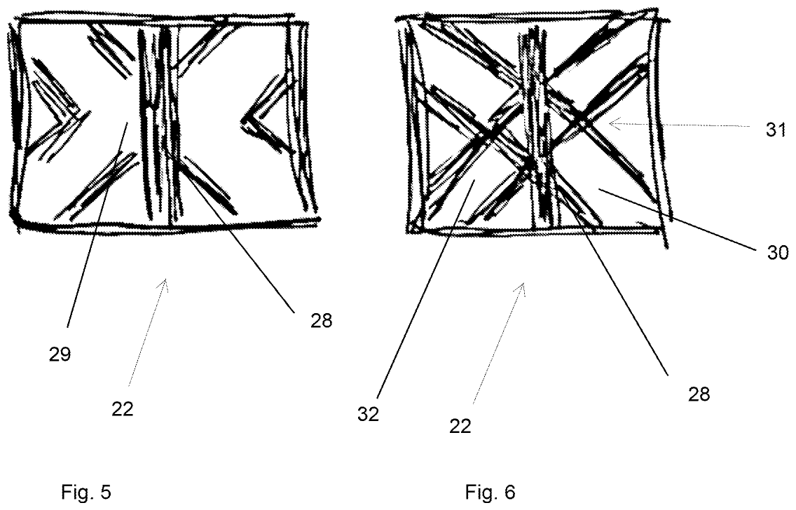

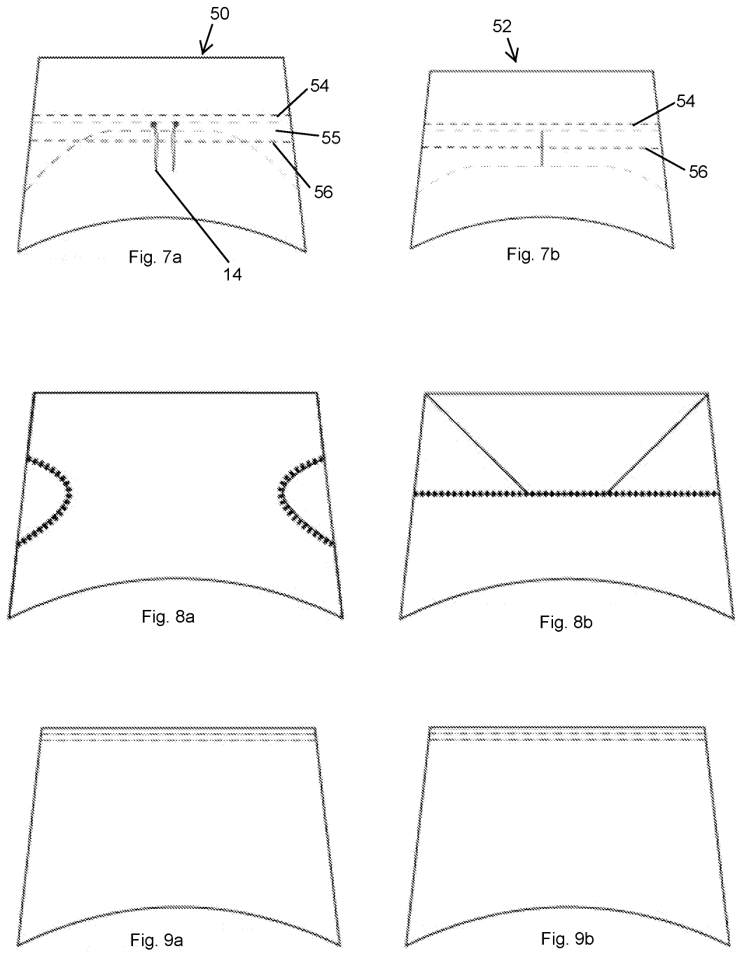

FIGS. 7a-9b illustrate a preferred embodiment of the outer layer of the waistband (FIGS. 7a and 7b) being the waistband outside layer and drawstring layer, the waistband neoprene layer (8a and 8b) and the waistband lining layer (FIGS. 9a and 9c) providing four layers of material in the three illustrated sections. FIG. 7a illustrates the front 50 of the waistband outer layer with FIG. 7b illustrating the back of the waistband outer layer. The waistband outer layer has a drawstring casing defined by stitching lines 54, 56 to provide for channel or casing 55. The term front and back refer to the positioning of the layers on a human torso, with the front and back layers preferably being continuous and depicted separately for illustrative purposes. The outer layer provides a draw string casing and serves to compress the neoprene layer. The neoprene drawstring shape is illustrated by lighter dashed lines. The outer layer is made of a ribbed knit compression material overlaying a neoprene material.

The waistband (or reinforced) layer is positioned between the outer layer and the lining layer. The waistband layer is made using a neoprene layer. The neoprene layer is illustrated in FIGS. 8a and 8b having a continuous construction, with seams provided for forming the shape of the material to form fit and support a human torso, and preferably a female human torso, with dark lines representing flat seams.

The innermost layer is depicted in FIGS. 9a and 9b. This Waistband lining layer is provided with a 4-way stretch compression material.

Still other features and advantages of the presently disclosed and claimed inventive concept(s) will become readily apparent to those skilled in this art from the following detailed description describing preferred embodiments of the inventive concept(s), simply by way of illustration of the embodiments contemplated by carrying out the inventive concept(s). As will be realized, the inventive concept(s) is capable of modification in various obvious respects all without departing from the inventive concept(s). Accordingly, the drawings and description of the preferred embodiments are to be regarded as illustrative in nature, and not as restrictive in nature.

While certain exemplary embodiments are shown in the Figures and described in this disclosure, it is to be distinctly understood that the presently disclosed inventive concept(s) is not limited thereto but may be variously embodied to practice within the scope of this disclosure. From the foregoing description, it will be apparent that various changes may be made without departing from the spirit and scope of the disclosure as defined herein.

I claim:

1. A lower body support and compression garment, said lower body support and compression garment comprising:
a multi layered waistband having a front side and a back side, wherein said multi layered waistband has an inner layer and an outer layer, said multi layered waistband is attached to the remainder of a lower body garment;
wherein said inner layer of said multi layered waistband is comprised of a compression material, wherein said inner layer has a compression and support structure, said compression and support structure including at least one vertical support structure positioned substantially in the middle of said back and/or front side of said inner layer, said compression and support structure is configured to provide compression and support to targeted areas of a wearer;
wherein said outer layer of said multi layered waistband is comprised of the same material as the remainder of said lower body garment, wherein said outer layer and said lower body garment are made from an antimicrobial, moisture wicking material or acceptable material of similar nature.

2. The lower body garment of claim 1 wherein said compression and support structure of said inner layer is designed as an X pattern, wherein said X pattern is configured to provide compression and support to the targeted areas.

3. The lower body garment of claim 2 wherein said compression and support structure further comprises a lower compression and support structure configured to circumvolve said inner layer, wherein said lower compression and support structure is configured to provide additional support to the targeted areas.

4. The lower body garment of claim 2 wherein there is a plurality of said vertical support structures.

5. The lower body garment of claim 1 wherein said compression and support structure of said inner layer is designed with angled compressive support structures, wherein said angled compressive support structures are configured to provide compression and support to the targeted areas.

6. The lower body garment of claim 5 wherein said compression and support structure further comprises a lower compression and support structure configured to circumvolve said inner layer, wherein said lower compression and support structure is configured to provide additional support to the targeted areas.

7. The lower body garment of claim 5 wherein there is a plurality of said vertical support structures.

8. The lower body garment of claim 1 wherein said multi layered waistband further comprises a drawstring configured to provide additional support.

9. The lower body garment of claim 1 wherein said multi layered waistband comprises four layers, said layers comprising a waistband outside layer, a drawstring layer, a Waistband (Reinforced) layer, and a waistband lining layer.

10. The lower body garment of claim 9 wherein said waistband outside layer comprises a ribbed knit compression material, wherein said drawstring layer comprises a neoprene and a compression material, wherein said waistband layer comprises a neoprene, and wherein said waistband lining layer comprises a 4-way stretch compression material.
